(19) United States
(12) Patent Application Publication
(10) Pub. No.: US 2025/0090907 A1
(43) Pub. Date: Mar. 20, 2025

(54) AUTOMATIC WORKOUT ADJUSTMENT BASED ON HEART RATE VARIABILITY DATA

(71) Applicant: VeloPro, Inc., Portland, OR (US)

(72) Inventors: Adnan KADIR, Portland, OR (US); Robert C. VIZZA, San Francisco, CA (US)

(21) Appl. No.: 18/889,062

(22) Filed: Sep. 18, 2024

Related U.S. Application Data
(60) Provisional application No. 63/583,702, filed on Sep. 19, 2023.

(51) Int. Cl. A63B 24/00; A63B 71/06; G16H 20/30; G16H 50/30

(52) U.S. Cl. CPC A63B 24/0075; A63B 71/0622; G16H 20/30; G16H 50/30; A63B 2024/0065; A63B 2024/0068; A63B 2230/065

(57) ABSTRACT

In some examples, a system for dynamically adjusting one or more workout outputs comprises a heart rate monitor operatively coupled to an automatic workout generator. In some examples, a method of altering one or more workout outputs comprises receiving, by a user interface, one or more workout inputs provided by a user and generating one or more workout outputs based on the one or more workout inputs. In some examples, the method further comprises dynamically altering at least one of the one or more workout outputs in response to the determined physical readiness indicator of the user. In some examples, the method further comprises displaying the one or more workout outputs to the user. In some examples, the physical readiness indicator is determined by analyzing heart rate variability data.

### CROSS-REFERENCES

The following applications and materials are incorporated herein, in their entireties, for all purposes: U.S. Provisional Patent Application Ser. No. 63/583,702, filed Sep. 19, 2023.

### FIELD

This disclosure relates to systems and methods for automatic workout adjustment. More specifically, the disclosed embodiments relate to systems and methods for automatically adjusting a workout based on heart rate variability data.

### INTRODUCTION

Heart rate variability (HRV) data has gained widespread use in the fitness and sports industry, with device manufacturers providing HRV insights that offer valuable information about an athlete's autonomic nervous system and physical readiness. However, current exercise programs lack a systematic approach to incorporating HRV insights and determining real-time workout adjustments based on these HRV insights.

### SUMMARY

The present disclosure provides systems, apparatuses, and methods relating to automatic workout adjustment based on heart rate variability data.

In some examples, a method of adjusting one or more workout outputs comprises: receiving, by a user interface, one or more workout inputs provided by a user; generating one or more workout outputs based on the one or more workout inputs; based on a determined physical readiness indicator, dynamically altering at least one of the one or more workout outputs in response to the determined physical readiness indicator of the user; and displaying the one or more workout outputs to the user.

In some examples, non-transitory computer-readable storage media comprising computer executable software that when executed, directs a computing device to perform steps comprising: receiving, by a user interface, one or more workout inputs provided by a user; generating one or more workout outputs based on the one or more workout inputs; based on a physical readiness category of the user, dynamically altering at least one workout output of the one or more workout outputs; and providing the one or more workout outputs to the user.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a schematic diagram of a system for automatically adjusting a workout based on heart rate variability data in accordance with aspects of the present disclosure.

FIG. 2 is a flowchart depicting steps of an illustrative method for automatically adjusting a workout based on heart rate variability data according to the present disclosure.

FIG. 3 is a schematic diagram of a data processing system in accordance with aspects of the present disclosure.

FIG. 4 is a schematic diagram of a network data processing system in accordance with aspects of the present disclosure.

### DETAILED DESCRIPTION

Various aspects and examples of systems and methods for automatically adjusting a workout based on heart rate variability data are described below and illustrated in the associated drawings. Unless otherwise specified, a system for automatically adjusting a workout based on heart rate variability data in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Comprising,” “including,” and “having” (and conjugations thereof) are used interchangeably to mean “including but not necessarily limited to,” and are open-ended terms not intended to exclude additional, unrecited elements or method steps.

Terms such as “first,” “second,” and “third” are used to distinguish or identify various members of a group or the like, and are not intended to show serial or numerical limitation.

“AKA” means “also known as,” and may be used to indicate an alternative or corresponding term for a given element or elements.

“Processing logic” describes any suitable device(s) or hardware configured to process data by performing one or more logical and/or arithmetic operations (e.g., executing coded instructions). For example, processing logic may include one or more processors (e.g., central processing units [CPUs] and/or graphics processing units [GPUs]), microprocessors, clusters of processing cores, FPGAs (field-programmable gate arrays), artificial intelligence (AI) accelerators, digital signal processors (DSPs), and/or any other suitable combination of logic hardware.

“Providing,” in the context of a method, may include receiving, obtaining, purchasing, manufacturing, generating, processing, preprocessing, and/or the like, such that the object or material provided is in a state and configuration for other steps to be carried out.

### Overview

In general, a system for automatically adjusting a workout based on heart rate variability (HRV) data integrates HRV insights with an automatic workout generator to generate and update personalized exercise regimens configured to adapt to a user's daily physical readiness. In some examples, the HRV insights are utilized in tandem with a user's measured functional threshold power (FTP) in generating and updating the exercise regimens.

In general, a user may provide the system with one or more desired fitness goals and based on the user's desired goal(s), the system is configured to automatically generate a periodized workout regimen tailored to the desired fitness goal(s). A workout regimen, as described herein, corresponds to a set of instructions to perform one or more activities, which is provided to and/or displayed to the user. In some examples, these instructions comprise activity intensity, target heart rate, instructions to periodically vary a power, activity intensity, activity duration, a target distance traveled, and/or the like. In some examples, the desired fitness goal(s) may comprise a desired degree of power, strength, endurance, and/or athletic skill. Accordingly, the system may generate a workout regimen configured to provide strength training, endurance training, and/or skill development. The workout regimen may include one or more various workouts configured to aid the user in reaching the desired fitness goal(s). In some examples, the desired fitness goal(s) may comprise a desired degree of fitness for a specific event and/or event type, such as an event having a specified distance, duration, intensity, activity type, and/or the like. In some examples, the desired fitness goal(s) comprise a target functional threshold power (FTP). In some examples, the workout regimen comprises a daily schedule of workouts and rest days. Systems and methods described herein are suitable for use with a variety of athletic activities, such as cycling, running, swimming, rowing, paddling, sailing, walking, weightlifting, and/or the like.

The system is configured to periodically receive HRV data (e.g., hourly, daily, weekly, etc.), from a device capable of measuring the HRV data, such as a wearable HRV monitor, (e.g., worn by the user). In some examples, the heart rate measurement device comprises a wearable heart rate sensor (e.g., a sensor configured to measure heart rate data) and a coupled computing device configured to calculate heart rate variability insights based on the measured heart rate data. As an individual's heart rate is regulated by the autonomic nervous system, HRV data provide insight into an individual's physiological state. Increased HRV is associated with a well-functioning autonomic system and ability to adapt to stress, while decreased HRV can be indicative of fatigue, overtraining, or other health issues. Accordingly, monitoring HRV can help optimize training loads and avoid overtraining. More specifically, acute changes in an athlete's HRV can signal whether they are coping well with training loads or require more recovery. Furthermore, chronic reduction in HRV levels may be a marker of maladaptation to training.

In general, the HRV data utilized by the system include root-mean-square of successive differences (RMSSD) data and/or standard deviation of normal-to-normal intervals (SDNN) data. The HRV data may be collected continuously, daily, weekly, and/or otherwise to track long-term trends. In some examples, the heart rate measurement device is configured to continuously record and transmit HRV metrics, such as interbeat intervals, heart rate variability indexes, and other relevant biometric data. In some examples, the heart rate measurement device is configured to process the raw HRV data and calculate HRV insights. In some examples, the HRV insights correspond to a normalized heart rate variability, presented as a point on the numerical scale. In some examples, the HRV insights may be categorized by the system into two or more physical readiness indicators and/or categories, characterizing the user's physiological state (e.g., well-rested, slightly fatigued, severely fatigued, etc.). In some examples, the system categorizes the HRV insights into the two or more physical readiness indicators or categories based on the normalized heart rate variability. In some examples, the system compares the HRV insights with one or more threshold physical readiness levels and assigns the user a physical readiness indicator and/or category based on the comparison of the physical readiness of the user to the one or more threshold physical readiness levels.

Before each scheduled workout session, the system is configured to analyze the user's HRV data to determine their physical readiness for engaging in the respective workout session and, if necessary, adjust the workout accordingly. For example, if the HRV data indicate the user is well-rested, the predefined workout may be assigned (e.g., displayed, provided) without alteration. Additionally or alternatively, if the HRV data suggest slight fatigue, the system may adjust one or more workout parameters, such as reducing training intensity and/or volume, to accommodate the current physiological state. Additionally or alternatively, if the HRV data suggest slight fatigue, the system may modify a workout type assigned to the user, to accommodate the current physiological state. Additionally or alternatively, if the HRV data indicate severe fatigue, the system may replace the current workout with a rest day to facilitate proper recovery. Accordingly, throughout the training period, the system is configured to analyze HRV data, track workout performance, and assess progress towards the user's goals. In some examples, throughout the training period, the system incorporates further benchmark data, such as a measured functional threshold power (FTP) of the user, to further adjust the user's workout regimen.

Aspects of a system for automatically adjusting a workout based on heart rate variability data may be embodied as a computer method, computer system, or computer program product. Accordingly, aspects of the system for automatically adjusting a workout based on heart rate variability data may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the system for automatically adjusting a workout based on heart rate variability data may take the form of a computer program product embodied in a computer-readable medium (or media) having computer-readable program code/instructions embodied thereon.

In some examples, systems and methods according to the present teachings comprise non-transitory computer-readable storage media comprising computer executable software that when executed directed a computing device to perform any of the methods described herein.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of the system may be written in one or any combination of programming languages, including an object-oriented programming language (such as Java or C++), conventional procedural programming languages (such as C), and functional programming languages (such as Haskell). Mobile apps may be developed using any suitable language, including those previously mentioned, as well as Objective-C, Swift, C#, HTML5, and the like. The program code may execute entirely on a user's computer, partly on the user's computer, as a stand-alone software package, partly on the user's computer and partly on a remote computer or server, or entirely on the remote computer or server. In the latter scenario, the remote computer or server may be connected to the user's computer through any type of network, including a local area network (LAN) or a wide area network (WAN), and/or the connection may be made to an external computer (for example, through the Internet using an Internet Service Provider).

Aspects of the system for automatically adjusting a workout based on heart rate variability data may be described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array [FPGA], or other programmable data processing apparatus) to produce a machine, such that the instructions (e.g., machine-readable instructions), which execute via the processing logic, create means for implementing the functions/acts specified in the flowchart and/or block diagram block(s).

Additionally or alternatively, the computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed by the processing logic to produce a computer-implemented process, such that the executed instructions provide processes for implementing the functions/acts specified in the flowchart and/or block diagram block(s).

Each flowchart and/or block diagram in the Figures is intended to illustrate the architecture, functionality, and/or operation of possible implementations of systems, methods, and computer program products according to aspects of the system for automatically adjusting a workout based on heart rate variability data. In this regard, each function/act and block diagram block may represent a module, segment, or portion of code, which comprises one or more executable instructions for implementing the specified logical function(s). In some implementations, the function(s) noted in the act/block may occur out of the order illustrated in the Figures. For example, two acts/blocks shown in succession may, in fact, be executed substantially concurrently, or sometimes the acts/blocks may be executed in the reverse order, depending upon the functionality involved. Each act/block and/or combination of acts/blocks may be implemented by special purpose hardware-based systems (or combinations of special purpose hardware and computer instructions) that perform the specified functions or steps.

### Examples, Components, and Alternatives

The following subsections describe selected aspects of an illustrative system for automatically adjusting a workout based on heart rate variability data, as well as related systems and/or methods. The examples in the following subsections are intended for illustration and should not be interpreted as limiting the scope of the present disclosure. Each subsection may include one or more distinct embodiments or examples, and/or contextual or related information, function, and/or structure.

### A. System for Automatically Adjusting a Workout Based on Heart Rate Variability Data

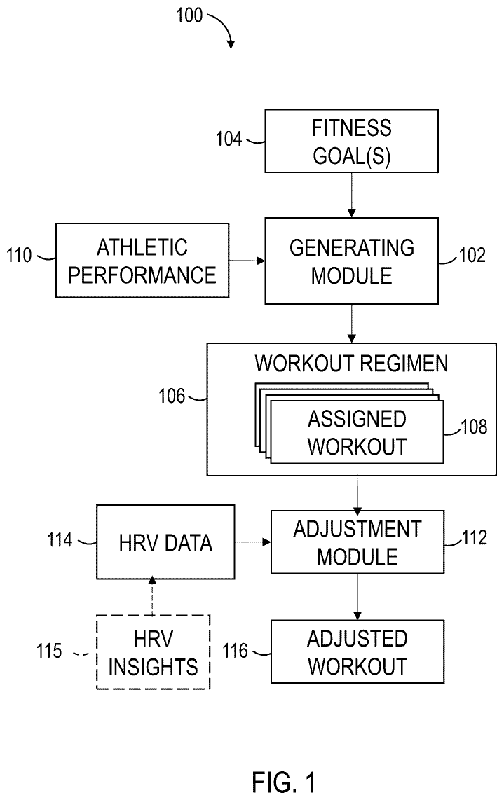

As shown in FIG. 1, this subsection describes an illustrative system 100 for automatically adjusting a workout based on heart rate variability data. System 100 is an example of the system for automatically adjusting a workout based on heart rate variability data described above. Accordingly, system 100 utilizes HRV data to automatically adjust a workout to adapt to a user's daily physical readiness. System 100 is suitable for use with a variety of athletic activities, such as cycling, running, swimming, rowing, paddling, sailing, walking, weightlifting, and/or the like.

With continued reference to FIG. 1, a user may provide a generating module 102 with one or more desired fitness goal(s) 104. Generating module 102 of system 100 is configured to automatically generate a periodized workout regimen 106 having a plurality of assigned workouts 108 tailored to the desired fitness goal(s). In some examples, generating module 102 may additionally utilize historical athletic performance data 110, such as functional threshold power (FTP) data, in generating workout regimen 106. In some examples, the user provides the generating module with initial FTP data, which is utilized to calculate goal FTP levels corresponding to the one or more desired fitness goals. In some examples, the user provides the generating module with one or more workout inputs, such as the one or more desired fitness goals, a desired training frequency, a desired training time, and/or the like.

In some examples, desired fitness goal(s) 104 may comprise a desired degree of power, strength, endurance, and/or athletic skill. For example, a desired degree of strength may correspond to a desired functional threshold power (FTP), a desired speed over a short distance, and/or the like. Similarly, a desired degree of endurance may correspond to a desired speed over a medium or long distance, and/or the like. Accordingly, generating module 102 may generate workout regimen 106 to be configured to provide strength training, endurance training, and/or skill development. Workout regimen 106 may include one or more various workouts configured to aid the user in reaching their desired fitness goal(s). In some examples, workout regimen 106 comprises a daily schedule of assigned workouts 108 and rest days. In some examples, desired fitness goal(s) 104 comprise event parameters, such as an event date, an event distance, an event type, and/or the like. Accordingly, generating module 102 may generate workout regimen 106 to correspond to a user achieving a desired degree of fitness at a desired date. For example, a user training for an endurance event may provide generating module 102 with an event date for the endurance event and indicate that the event type is an endurance event. Generating module 102 may subsequently generate a training plan configured to maximize the user's degree of endurance on the event date. Similarly, a user training for a strength-based or power-based event may provide generating module 102 with an event date for the strength-based or power-based event and indicate that the event type is a strength-based or power-based event. Generating module 102 may subsequently generate a training plan configured to maximize the user's degree of power (i.e., functional threshold power) on the event date. Generated workouts 108, as described herein, may correspond to sets of instructions to perform one or more activities, which are provided to and/or displayed to the user. In some examples, these instructions comprise activity intensity, target heart rate, instructions to periodically vary an activity intensity, activity duration, a target distance traveled, and/or the like. In some examples, generated workouts 108 may incorporate instructions to repeat specific actions, utilize specific training equipment, perform actions under certain conditions (e.g., hills, etc.), and/or the like. In some examples, the generated workouts correspond to one or more workout types, as defined, for example, by the Coggan system, such as active recovery, endurance, tempo, threshold, VO₂ max, anaerobic, benchmark, and/or the like.

The generated workouts 108 may include a periodized training plan configured to generate and/or utilize a specific Training Load (TL) configured to aid in achieving the desired fitness goal(s), such as a desired completion time for a specified event, a desired fitness level for a specified event, a desired strength, endurance, or skill level, and/or the like. TL corresponds to an expected performance improvement of a user. The generating module utilizes the TL in a quantified fitness model. As an illustrative example, generating module 102 may utilize a coupled set of differential equations to quantify the dynamics of fitness and fatigue, such as:

$$dB / dt = {k}_{1} - {k}_{2} P$$

$$dP / dt = {k}_{4} TL - {k}_{3} P$$

However, generating module 102 may additionally or alternatively utilize another suitable analytical model for quantifying fitness and fatigue. In the above set of illustrative differential equations, fitness (B) refers to the user's physiological adaptation to TL, and fatigue (P) refers to a short-term depletion of performance capacity induced by training. Accordingly, generating module 102 estimates changes in fitness and fatigue based on the TL of the generated workouts.

Model parameters k₁-k₄ are used by the system 100 to adapt the workout regimen to the user. In some examples, the model parameters are learned, for example through the use of one or more machine-learning models. For example, the model parameters may be learned with one or more supervised and/or unsupervised learning models such as regression models, neural networks, Bayesian networks, clustering models, and reinforcement learning models. In some examples, the model parameters are determined through the use of a gradient descent model in conjunction with historical and continued athletic performance 110 of the user.

Accordingly, the model parameters can be optimized to the user by fitting the model to the user's TL data, thereby enabling quantification of the fitness and fatigue response to past training. Projecting forward, generating module 102 can prescribe training loads which optimize fitness while avoiding excessive fatigue and overtraining. Heart rate variability data approximate a present training load of a user, facilitating the adjustment of generating module 102 based on a user's physiological data.

System 100 is configured to periodically receive HRV data 114 (e.g., continuously, hourly, daily, weekly, monthly), from a device capable of measuring the HRV data, such as a wearable heart rate monitor, (e.g., worn by the user, such as a watch, ring, chest strap, wristband, and/or the like), a processor coupled to a wearable heart rate monitor, one or more workout machines (e.g., a treadmill, exercise bike, etc.) incorporating heart rate monitors, and/or the like. In some examples, the HRV data utilized by the system include root-mean-square of successive differences (RMSSD) data and/or standard deviation of normal-to-normal intervals (SDNN) data. The HRV data may be collected continuously, daily, weekly, and/or otherwise to track long-term trends. In some examples, the heart rate measurement device is configured to continuously record and transmit HRV metrics, such as interbeat intervals, heart rate variability indexes, and other relevant biometric data. In some examples, the heart rate measurement device is configured to transmit raw HRV data to a user. In some examples, system 100 further comprises a power meter configured to periodically measure a functional threshold power of the user. In some examples, the measured functional threshold power is further utilized in adjusting the user's workout regimen.

In some examples, the heart rate measurement device is configured to process the raw HRV data and calculate HRV insights. In some examples, the HRV insights correspond to a normalized heart rate variability, presented as a point on a numerical scale. In some examples, the HRV insights and/or the normalized heart rate variability are provided to the system by the heart rate measurement device.

HRV data 114 are categorized by the system into a plurality of readiness indicators and/or categories (e.g., well-rested, slight fatigued, severe fatigued, etc.), configured to characterize the user's readiness for the assigned workout of the workout regimen 106. Accordingly, before each assigned workout 108, an adjustment module 112 is configured to analyze the most recent HRV data 114 and/or HRV insights in concert with assigned workout 108 to determine a physical readiness for engaging in the respective workout session and, if necessary, adjust the workout accordingly to produce an adjusted workout 116. In some examples, the system categorizes the HRV insights into the two or more physical readiness indicators or categories based on the normalized heart rate variability. In some examples, the system compares the HRV insights with one or more threshold physical readiness levels and assigns the user a physical readiness indicator and/or category based on the comparison of the physical readiness of the user to the one or more threshold physical readiness levels.

In some examples, the system receives HRV data from the heart rate measurement device in the form of a point on a numerical scale. For example, the user's HRV data may be represented on a scale of 1-5, 1-10, 1-100, and/or the like. Accordingly, in some examples, the system categorizes the HRV insights into the two or more physical readiness indicators based on the numerical scale utilized by the heart rate measurement device.

For example, if HRV data 114 indicate the user is well-rested, assigned workout 108 may be provided to the user without alteration. Additionally or alternatively, if HRV data 114 suggest slight fatigue, system 100 may adjust the training load of assigned workout 108, such as reducing training intensity or volume, to accommodate the slightly fatigued state. In some examples, reducing training intensity or volume includes reducing an assigned distance, duration, intensity, number of repetitions, and/or the like. Additionally or alternatively, if HRV data 114 indicate severe fatigue, system 100 may replace assigned workout 108 with a new, less intense, adjusted workout (or a rest day) to facilitate proper recovery. For example, system 100 may replace a tempo workout with an endurance workout. In some examples, if HRV data 114 indicate severe fatigue, system 100 may both replace assigned workout 108 with a less intense workout and reduce a training intensity and/or volume. Accordingly, throughout the training period, the system is configured to analyze HRV data, track workout performance, and assess progress towards the user's desired fitness goal(s). In some examples, the system incorporates insights gained from benchmark workouts, such as FTP checks performed using a power meter.

### B. Illustrative Method

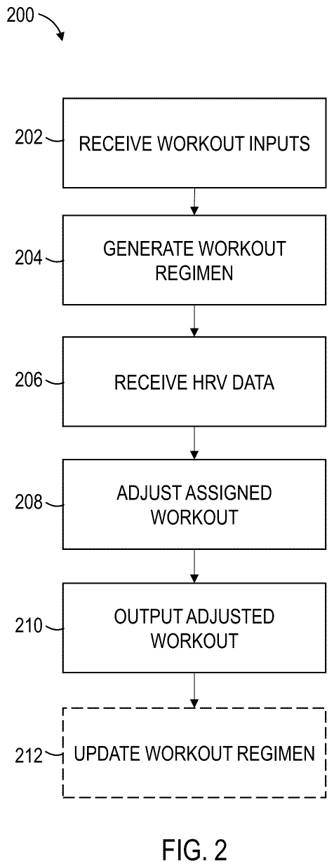

This subsection describes steps of an illustrative method 200 for automatically adjusting a workout based on heart rate variability data; see FIG. 2. Aspects of the system for automatically adjusting a workout based on heart rate variability data described above may be utilized in the method described below. Where appropriate, reference may be made to components and systems that may be used in carrying out each step of method 200. These references are for illustration, and are not intended to limit the possible ways of carrying out any particular step of the method.

FIG. 2 is a flowchart illustrating steps performed in an example method, and may not recite the complete method or all steps of the method. Although various steps of method 200 are described below and depicted in FIG. 2, the steps need not necessarily all be performed, and in some cases may be performed simultaneously or in a different order than the order shown.

At step 202 of method 200, one or more workout inputs are received, from a user. The workout inputs may comprise desired fitness goals, such as a desired degree of strength, endurance, and/or athletic skill, or other suitable fitness goal such as weight loss, etc. In some examples, the desired fitness goals comprise event parameters, such as an event date, an event distance, an event type, and/or the like. In some examples, the workout inputs comprise a desired training frequency, a desired training time, a desired training load, and/or the like. In some examples, the one or more workout inputs are received by a user interface, such as a display, mobile device, tablet, personal computer, remote, and/or the like.

At step 204 of method 200, a periodized workout regimen is generated based on the workout inputs. The workout regimen may include one or more various workouts configured to aid the user in reaching their desired fitness goal(s). For example, the system may generate a workout regimen configured to provide strength training, endurance training, and/or skill development. In some examples, the workout regimen comprises a daily schedule of workouts and rest days. In some examples, the generated workouts correspond to sets of instructions to perform one or more activities, which are provided to and/or displayed to the user. In some examples, these instructions comprise activity intensity, power levels, target heart rate, instructions to periodically vary an activity intensity, instructions to periodically vary a power level, activity duration, a target distance traveled, and/or the like. In some examples, the workout regimen may incorporate instructions to repeat specific actions, utilize specific training equipment, perform actions under certain conditions (e.g., hills, etc.), and/or the like. In some examples, the workout regimen corresponds to one or more workout types, as defined, for example, by the Coggan system, such as active recovery, endurance, tempo, threshold, VO₂ max, anaerobic, benchmark, and/or the like.

At step 206 of method 200, HRV data are periodically received (e.g., continuously, hourly, daily, weekly, monthly, and/or the like), for example, from a device capable of measuring heart rate variability such as a wearable heart rate monitor, a processor coupled to a wearable heart rate monitor, one or more workout machines incorporating heart rate monitors, and/or the like. In some examples, the HRV data comprise HRV metrics, such as interbeat intervals, heart rate variability indexes, and other relevant biometric data. In some examples, the HRV data may comprise processed HRV data referred to as “HRV insights.”

For example, HRV insights may comprise categorized readiness indicators characterizing the user's physiological state, such as a well-rested state, a slightly fatigued state, a severely fatigued state, etc. In some examples, the HRV insights correspond to a normalized heart rate variability, presented as a point on a numerical scale. In some examples, the HRV insights and/or the normalized heart rate variability are provided to the system by the heart rate measurement device. In some examples, step 206 of method 200 includes categorizing and/or determining a physical readiness category and/or indicator from a set of physical readiness categories and/or indicators. Accordingly, in some examples, step 206 of method 200 includes comparing the HRV data and/or HRV insights with one or more threshold physical readiness values, optionally presented as points on the numerical scale, and determining the physical readiness indicator and/or physical readiness category based on the comparison of the HRV data with the one or more threshold physical readiness values.

At step 208 of method 200, the respective workout assigned from the periodic workout regimen is adjusted (i.e., dynamically altered) based on the HRV data received at step 206. In some examples, step 208 includes analyzing the received HRV data to determine the user's physical readiness for engaging in the respective assigned workout and, if necessary, adjust the assigned workout accordingly. More specifically, the workout regimen may be adjusted based on the user's determined physical readiness indicator and/or physical readiness category. For example, if the HRV data indicate the user is well-rested, the predefined workout may be assigned with minor (or no) alterations. Additionally or alternatively, if the HRV data suggest slight fatigue, the system may adjust one or more workout parameters or output parameters, such as a workout intensity or workout duration, e.g., by reducing training intensity or volume, to accommodate the current physiological state of the user. In some examples, reducing training intensity or volume includes reducing an assigned distance, duration, intensity, number of repetitions, and/or the like. Additionally or alternatively, if the HRV data indicate severe fatigue, step 208 may replace the respective assigned workout with a new, less intense, adjusted workout to facilitate proper recovery. For example, step 208 may replace a tempo workout with an endurance workout. In some examples, if the HRV data indicate severe fatigue, step 208 may both replace the respective assigned workout with a less intense workout and reduce a training intensity and/or volume. Additionally or alternatively, if the HRV insights indicate severe fatigue, the system may replace the assigned workout with a rest day to facilitate proper recovery. In some examples, the system incorporates insights gained from benchmark workouts, such as FTP checks performed using a power meter.

At step 210 of method 200, the adjusted workout is output. In some examples, outputting the adjusted workout includes sending instructions for the adjusted workout to a digital display device. Additionally or alternatively, outputting the adjusted workout may include printing the adjusted workout and/or any other suitable output method for providing the adjusted workout to the user.

At optional step 212 of method 200, the workout regimen is updated (e.g., changed) based on historical HRV data, historical athletic performance data, fitness progress data, and/or other historical changes to the user's fitness levels. In some examples, optional step 212 includes analyzing historical HRV data and historical workout performances and comparing each to expected values at a given progression through the workout regimen. In some examples, optional step 212 of method 200 comprises periodically reassessing the user's physical readiness and further adjusting the one or more workout outputs accordingly.

### C. Data Processing System

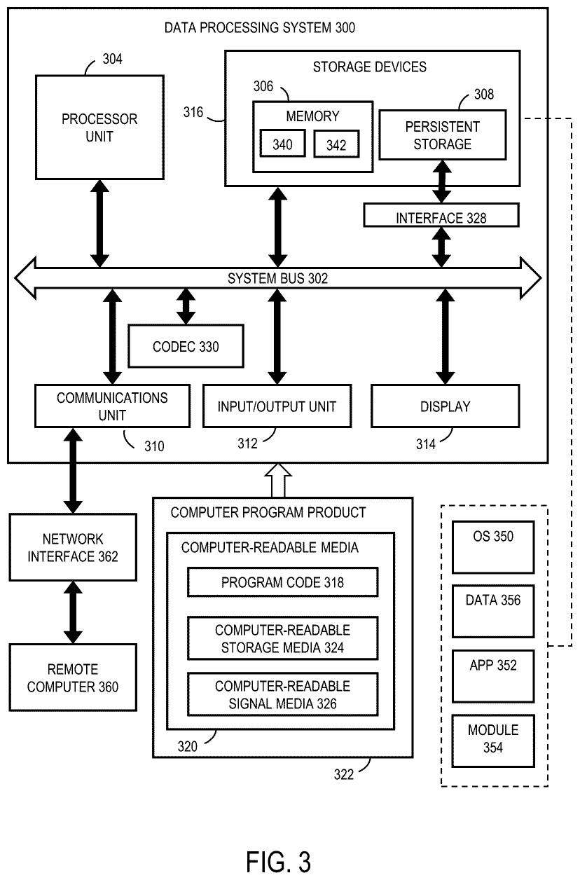

As shown in FIG. 3, this subsection describes a data processing system 300 (AKA a computer, computing system, and/or computer system) in accordance with aspects of the present disclosure. In the example of FIG. 3, data processing system 300 is an illustrative data processing system suitable for implementing aspects of the system for automatically adjusting a workout based on heart rate variability data, as described herein. More specifically, in some examples, devices that are embodiments of data processing systems (e.g., smartphones, tablets, personal computers) may be utilized in generating workout regimens, analyzing HRV data, characterizing fitness and/or readiness, and/or updating workouts.

In this illustrative example, data processing system 300 includes a system bus 302 (also referred to as “communications framework”). System bus 302 may provide communications between a processor unit 304 (also referred to as a “processor” or “processors”), a memory 306, a persistent storage 308, a communications unit 310, an input/output (I/O) unit 312, a codec 330, and/or a display 314. Memory 306, persistent storage 308, communications unit 310, I/O unit 312, display 314, and codec 330 are examples of resources that may be accessible by processor unit 304 via system bus 302.

Processor unit 304 runs instructions that may be loaded into memory 306. Processor unit 304 may comprise a number of processors, a multi-processor core, and/or a particular type of processor(s) (e.g., a central processing unit [CPU], graphics processing unit [GPU], etc.), depending on the particular implementation. Furthermore, processor unit 304 may be implemented using a number of heterogeneous processor systems, in which a main processor is present with secondary processors on a single chip. As another illustrative example, processor unit 304 may be a symmetric multi-processor system containing multiple processors of the same type.

Memory 306 and persistent storage 308 are examples of storage devices 316. A storage device may include any suitable hardware capable of storing information (e.g., digital information), such as data, program code in functional form, and/or other suitable information, either on a temporary basis or a permanent basis.

Storage devices 316 also may be referred to as computer-readable storage devices or computer-readable media. Memory 306 may include a volatile memory 340 and a non-volatile memory 342. In some examples, a basic input/output system (BIOS) containing the basic routines to transfer information between elements within the data processing system 300, such as during start-up, may be stored in non-volatile memory 342. Persistent storage 308 may take various forms, depending on the particular implementation.

Persistent storage 308 may contain one or more components or devices. For example, persistent storage 308 may include one or more devices such as a magnetic disk drive (AKA a hard disk drive [HDD]), solid state disk (SSD), floppy disk drive, tape drive, Jaz drive, Zip drive, flash memory card, memory stick, and/or the like, or any combination of these. One or more of these devices may be removable and/or portable, (e.g., a removable hard drive). Persistent storage 308 may include one or more storage media, separately or in combination with other storage media, including an optical disk drive, such as a compact disk ROM device (CD-ROM), CD-recordable drive (CD-R Drive), CD-rewritable drive (CD-RW Drive), and/or a digital versatile disk ROM drive (DVD-ROM). To facilitate connection of the persistent storage 308 to system bus 302, a removable or non-removable interface is typically used, such as interface 328.

I/O unit 312 allows for input and output of data with other devices that may be connected to data processing system 300 (i.e., input devices and output devices). For example, an input device may include one or more pointing and/or information-input devices such as a keyboard, a mouse, a trackball, stylus, touch pad or touch screen, microphone, joystick, game pad, satellite dish, scanner, TV-tuner card, digital camera, digital video camera, web camera, and/or the like. These and other input devices may connect to processor unit 304 through system bus 302 via interface port(s). Suitable interface port(s) may include, for example, a serial port, a parallel port, a game port, and/or a universal serial bus (USB) port.

One or more output devices may use some of the same types of ports, and in some cases the same actual ports, as the input device(s). For example, a USB port may be used to provide input to data processing system 300 and to output information from data processing system 300 to an output device. One or more output adapters may be provided for certain output devices (e.g., monitors, speakers, and printers, among others) which require special adapters. Suitable output adapters may include video and sound cards that provide a means of connection between the output device and system bus 302. Other devices and/or systems of devices may provide both input and output capabilities, such as remote computer 360. Display 314 may include any suitable human-machine interface or other mechanism configured to display information to a user, e.g., a CRT, LED, or LCD monitor or screen, etc.

Communications unit 310 refers to any suitable hardware and/or software employed to provide communications with other data processing systems or devices. While communications unit 310 is shown inside data processing system 300 in the example of FIG. 3, in some examples, communications unit 310 may be at least partially external to data processing system 300. Communications unit 310 may include internal and external technologies, e.g., modems (including regular telephone-grade modems, cable modems, and DSL modems), ISDN adapters, and/or wired and wireless Ethernet cards, hubs, routers, etc. Data processing system 300 may operate in a networked environment, using logical connections to one or more remote computers 360. A remote computer 360 may include a personal computer (PC), a server, a router, a network PC, a workstation, a microprocessor-based appliance, a peer device, a smart phone, a tablet, another network note, and/or the like. Remote computer(s) 360 typically include many of the elements described relative to data processing system 300. Remote computer(s) 360 may be logically connected to data processing system 300 through a network interface 362, which is connected to data processing system 300 via communications unit 310. Network interface 362 encompasses wired and/or wireless communication networks, such as local-area networks (LAN), wide-area networks (WAN), and cellular networks. LAN technologies may include Fiber Distributed Data Interface (FDDI), Copper Distributed Data Interface (CDDI), Ethernet, Token Ring, and/or the like. WAN technologies include point-to-point links, circuit-switching networks (e.g., Integrated Services Digital networks [ISDN] and variations thereon), packet-switching networks, and Digital Subscriber Lines (DSL).

Codec 330 may include an encoder, a decoder, or both, comprising hardware, software, or a combination of hardware and software. Codec 330 may include any suitable device and/or software configured to encode, compress, and/or encrypt a data stream or signal for transmission and storage, and to decode the data stream or signal by decoding, decompressing, and/or decrypting the data stream or signal (e.g., for playback or editing of a video). Although codec 330 is depicted as a separate component in the example of FIG. 3, codec 330 may be contained or implemented in memory, e.g., non-volatile memory 342.

Non-volatile memory 342 may include read only memory (ROM), programmable ROM (PROM), electrically programmable ROM (EPROM), electrically erasable programmable ROM (EEPROM), flash memory, and/or the like, or any combination of these. Volatile memory 340 may include random access memory (RAM), which may act as external cache memory. RAM may comprise static RAM (SRAM), dynamic RAM (DRAM), synchronous DRAM (SDRAM), double data rate SDRAM (DDR SDRAM), enhanced SDRAM (ESDRAM), and/or the like, or any combination of these.

Instructions for the data processing system, applications, and/or programs may be located in storage devices 316, which are in communication with processor unit 304 through system bus 302. In these illustrative examples, the instructions are in a functional form in persistent storage 308. These instructions may be loaded into memory 306 for execution by processor unit 304. Processes of one or more embodiments of the present disclosure may be performed by processor unit 304 using computer-implemented instructions, which may be located in a memory, such as memory 306.

These instructions are also referred to as program instructions, program code, computer-usable program code, or computer-readable program code executed by a processor in processor unit 304. The program code in different embodiments may be embodied on different physical or computer-readable storage media, such as memory 306 or persistent storage 308. Program code 318 may be located in a functional form on computer-readable media 320 that is selectively removable and may be loaded onto or transferred to data processing system 300 for execution by processor unit 304. Program code 318 and computer-readable media 320 form computer program product 322 in these examples. In one example, computer-readable media 320 may comprise computer-readable storage media 324 or computer-readable signal media 326.

Computer-readable storage media 324 may include, for example, an optical or magnetic disk that is inserted or placed into a drive or other device that is part of persistent storage 308 for transfer onto a storage device, such as a hard drive, that is part of persistent storage 308. Computer-readable storage media 324 also may take the form of a persistent storage, such as a hard drive, a thumb drive, or a flash memory, that is connected to data processing system 300. In some instances, computer-readable storage media 324 may not be removable from data processing system 300.

In these examples, computer-readable storage media 324 is a non-transitory, physical or tangible storage device used to store program code 318, rather than a medium that propagates or transmits program code 318. Computer-readable storage media 324 is also referred to as a computer-readable tangible storage device or a computer-readable physical storage device. In other words, computer-readable storage media 324 is media that can be held by a person.

Alternatively, program code 318 may be transferred to data processing system 300 (e.g., remotely over a network) using computer-readable signal media 326. Computer-readable signal media 326 may be, for example, a propagated data signal containing program code 318. For example, computer-readable signal media 326 may be an electromagnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 318 may be downloaded over a network to persistent storage 308 from another device or data processing system through computer-readable signal media 326 for use within data processing system 300. For instance, program code stored in a computer-readable storage medium in a server data processing system may be downloaded over a network from the server to data processing system 300. The computer providing program code 318 may be a server computer, a client computer, or some other device capable of storing and transmitting program code 318.

In some examples, program code 318 may comprise an operating system (OS) 350. OS 350, which may be stored on persistent storage 308, controls and allocates resources of data processing system 300. One or more applications 352 take advantage of the operating system's management of resources via program modules 354 and program data 356 stored on storage devices 316. OS 350 may include any suitable software system configured to manage and expose hardware resources of data processing system 300 for sharing and use by applications 352. In some examples, OS 350 provides application programming interfaces (APIs) that facilitate connection of different types of hardware, and/or provide applications 352 access to hardware and OS services. In some examples, certain applications 352 may provide further services for use by other applications 352, e.g., as is the case with so-called “middleware.” Aspects of present disclosure may be implemented with respect to various operating systems or combinations of operating systems.

The different components illustrated in FIG. 3 for data processing system 300 are not meant to provide architectural limitations to the manner in which different embodiments may be implemented. One or more embodiments of the present disclosure may be implemented in a data processing system that includes fewer components or additional components to and/or in place of those illustrated for data processing system 300. Other components shown in FIG. 3 may vary from the examples depicted. Different embodiments may be implemented using any hardware device or system capable of running program code. As one example, data processing system 300 may include organic components integrated with inorganic components, and/or may be comprised entirely of organic components (excluding a human being). For example, a storage device may be comprised of an organic semiconductor.

In some examples, processor unit 304 may take the form of a hardware unit having hardware circuits specifically manufactured or configured for a particular use, or to produce a particular outcome or process. This type of hardware may perform operations without a need for program code 318 to be loaded into a memory from a storage device to be configured to perform the operations. For example, processor unit 304 may be a circuit system, an application specific integrated circuit (ASIC), a programmable logic device, or some other suitable type of hardware configured (e.g., preconfigured or reconfigured) to perform a number of operations. With a programmable logic device, for example, the device is configured to perform the number of operations and may be reconfigured at a later time.

Examples of programmable logic devices include, a programmable logic array, a field programmable logic array, a field programmable gate array (FPGA), and other suitable hardware devices. With this type of implementation, executable instructions (e.g., program code 318) may be implemented as hardware, e.g., by specifying an FPGA configuration using a hardware description language (HDL) and then using a resulting binary file to configure, or reconfigure, the FPGA.

In another example, data processing system 300 may be implemented as an FPGA-based (or in some cases ASIC-based), dedicated-purpose set of state machines (e.g., Finite State Machines [FSM]), which may allow critical tasks to be isolated and run on custom hardware. Whereas a processor, such as a CPU, can be described as a shared-use, general-purpose state machine that executes instructions provided to it, FPGA-based state machines are constructed for a special purpose, and may execute hardware-coded logic without sharing resources. Such data processing systems are often utilized for safety-related and mission-critical tasks.

In still another illustrative example, processor unit 304 may be implemented using a combination of processors found in computers and hardware units. Processor unit 304 may have a number of hardware units and a number of processors that are configured to run program code 318. With this depicted example, some of the processes may be implemented in the number of hardware units, while other processes may be implemented in the number of processors.

In another example, system bus 302 may comprise one or more buses, such as a system bus or an input/output bus. Of course, the system bus may be implemented using any suitable type of architecture that provides for a transfer of data between different components or devices attached to the system bus. System bus 302 may include several types of bus structures, including memory bus or memory controller, a peripheral bus or external bus, and/or a local bus using any variety of available bus architectures (e.g., Industrial Standard Architecture [ISA], Micro-Channel Architecture [MSA], Extended ISA [EISA], Intelligent Drive Electronics [IDE], VESA Local Bus [VLB], Peripheral Component Interconnect [PCI], Card Bus, Universal Serial Bus [USB], Advanced Graphics Port [AGP], Personal Computer Memory Card International Association bus [PCMCIA], Firewire [IEEE 1394], and Small Computer Systems Interface [SCSI]).

Additionally, communications unit 310 may include a number of devices that transmit data, receive data, or both transmit and receive data. Communications unit 310 may be, for example, a modem or a network adapter, two network adapters, or some combination thereof. Further, a memory may be, for example, memory 306, or a cache, such as that found in an interface and memory controller hub that may be present in system bus 302.

### D. Illustrative Distributed Data Processing System

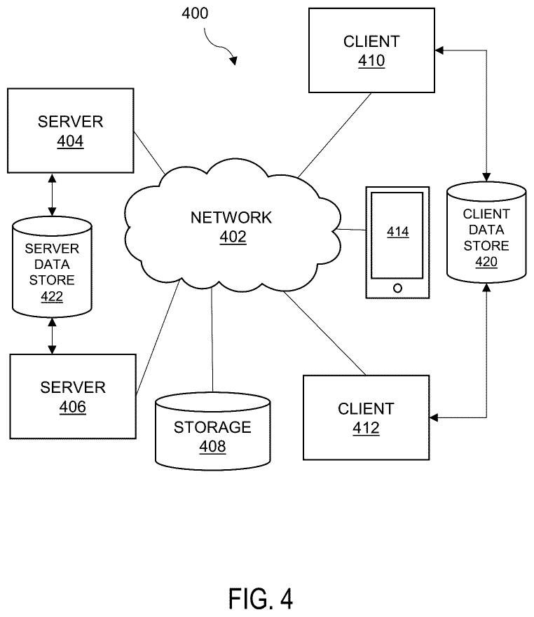

As shown in FIG. 4, this subsection describes a general network data processing system 400, interchangeably termed a computer network, a network system, a distributed data processing system, or a distributed network, aspects of which may be included in one or more illustrative embodiments of systems and/or methods for automatically adjusting a workout based on heart rate variability data. For example, communication between modules of the system 100, HRV monitoring devices, and/or other biometric sensors used in conjunction with the system 100, may be facilitated through the use of a network data processing system such as network system 400.

It should be appreciated that FIG. 4 is provided as an illustration of one implementation of such systems, and is not intended to imply any limitation with regard to environments in which different embodiments may be implemented. Many modifications to the depicted environment may be made.

Network system 400 is a network of devices (e.g., computers), each of which may be an example of data processing system 300 and other components. Network data processing system 400 may include network 402, which is a medium configured to provide communications links between various devices connected within network data processing system 400. Network 402 may include connections such as wired or wireless communication links, fiber optic cables, and/or any other suitable medium for transmitting and/or communicating data between network devices, or any combination thereof.

In the depicted example, a first network device 404 and a second network device 406 connect to network 402, as do one or more computer-readable memories or storage devices 408. Network devices 404 and 406 are each examples of data processing system 300, described above. In FIG. 4, devices of network system 400 are shown as server computers 404 and 406, which are in communication with one or more server data stores 422 that may be employed to store information local to server computers 404 and 406, among others. However, network devices may include, without limitation, one or more personal computers, mobile computing devices such as personal digital assistants (PDAs), tablets, and smartphones, handheld gaming devices, wearable devices, tablet computers, routers, switches, voice gates, servers, electronic storage devices, imaging devices, media players, and/or other networked-enabled tools that may perform a mechanical or other function. These network devices may be interconnected through wired, wireless, optical, and other appropriate communication links.

In addition, client electronic devices 410 and 412 and/or a client smart device 414, may connect to network 402. Each of these devices is an example of data processing system 300, described above regarding FIG. 3. Client electronic devices 410, 412, and 414 may include, for example, one or more personal computers, network computers, and/or mobile computing devices such as personal digital assistants (PDAs), smart phones, handheld gaming devices, wearable devices, and/or tablet computers, and the like. In FIG. 4, server computer 404 provides information, such as boot files, operating system images, and applications to one or more of client electronic devices 410, 412, and 414. Client electronic devices 410, 412, and 414 may be referred to as “clients” in the context of their relationship to a server, such as server computer 404. Client devices may be in communication with one or more client data stores 420, which may be employed to store information local to the clients (e.g., cookies and/or associated contextual information). Network data processing system 400 may include more or fewer servers and/or clients (or no servers or clients), as well as other devices not shown.

In some examples, first client electronic device 410 may transfer an encoded file to server 404. Server 404 can store the file, decode the file, and/or transmit the file to second client electronic device 412. In some examples, first client electronic device 410 may transfer an uncompressed file to server 404, and server 404 may compress the file. In some examples, server 404 may encode text, audio, and/or video information, and transmit the information via network 402 to one or more clients 410, 412, and 414.

Client smart device 414 may include any suitable portable electronic device capable of wireless communications and execution of software, such as a smartphone or a tablet. Generally speaking, the term “smartphone” may describe any suitable portable electronic device configured to perform functions of a computer, typically having a touchscreen interface, Internet access, and an operating system capable of running downloaded applications. In addition to making phone calls (e.g., over a cellular network), smartphones may be capable of sending and receiving emails, texts, and multimedia messages, accessing the Internet, and/or functioning as a web browser. Smart devices (e.g., smartphones) may include features of other known electronic devices, such as a media player, personal digital assistant, digital camera, video camera, and/or global positioning system. Such smart devices may be capable of connecting with other smart devices, computers, or electronic devices wirelessly, such as through near field communications (NFC), BLUETOOTH®, Wi-Fi, or mobile broadband networks. Wireless connectivity may be established among smart devices, smartphones, computers, and/or other devices to form a mobile network where information can be exchanged.

Data and program code located in network data processing system 400 may be stored in or on a computer-readable storage medium, such as network-connected storage device 408 and/or a persistent storage 308 of one of the network computers 404 and 406, as described above, and may be downloaded to a data processing system or other device for use. For example, program code may be stored on a computer-readable storage medium on server computer 404, and downloaded to client 410 over network 402 for use on client 410. In some examples, client data store 420 and server data store 422 reside on one or more storage devices 408 and/or 308.

Network data processing system 400 may be implemented as one or more of different types of networks. For example, network data processing system 400 may include an intranet, a local area network (LAN), a wide area network (WAN), or a personal area network (PAN). In some examples, network data processing system 400 includes the Internet, with network 402 representing a worldwide collection of networks and gateways that use the transmission control protocol/Internet protocol (TCP/IP) suite of protocols to communicate with one another. At the heart of the Internet is a backbone of high-speed data communication lines between major nodes or host computers. Thousands of commercial, governmental, educational and other computer systems may be utilized to route data and messages. In some examples, network 402 may be referred to as a “cloud.” In those examples, each server 404 may be referred to as a “cloud-computing node,” and client electronic devices may be referred to as “cloud consumers,” or the like. FIG. 4 is intended as an example, and not as an architectural limitation for any illustrative embodiments.

### E. Selected Embodiments and Claim Concepts

This subsection describes additional aspects and features of a system for automatically adjusting a workout based on heart rate variability data, presented without limitation as a series of paragraphs, some or all of which may be alphanumerically designated for clarity and efficiency. Each of these paragraphs can be combined with one or more other paragraphs, and/or with disclosure from elsewhere in this application in any suitable manner. Some of the paragraphs below may expressly refer to and further limit other paragraphs, providing without limitation examples of some of the suitable combinations.
A0. A method of adjusting one or more workout outputs, the method comprising: receiving, by a user interface, one or more workout inputs provided by a user; generating one or more workout outputs based on the one or more workout inputs; based on a determined physical readiness indicator, dynamically altering at least one of the one or more workout outputs in response to the determined physical readiness indicator of the user; and displaying the one or more workout outputs to the user.
A1. The method of paragraph A0, further comprising determining the physical readiness indicator of the user based on heart rate variability data.
A1.1. The method of paragraph A1, further comprising obtaining the heart rate variability data from a heart rate measurement device.
A1.2. The method of paragraph A1.1, wherein the heart rate measurement device comprises a heart rate sensor operatively coupled to a processor configured to calculate heart rate variability data from data collected by the heart rate sensor.
A2. The method of any of paragraphs A1 through A1.2, wherein determining the physical readiness indicator of the user further comprises:
   comparing the heart rate variability data with one or more threshold physical readiness values; and
   determining the physical readiness indicator based on the comparison of the heart rate variability data with the one or more threshold physical readiness values.
A2.1. The method of paragraph A2, wherein the analyzing the collected heart rate variability data to determine a physical readiness indicator of the user comprises normalizing the heart rate variability data to a numerical scale.
A2.1.1. The method of paragraph A2.1, wherein normalizing the heart rate variability data to a numerical scale is performed by a/the wearable device.
A2.2. The method of any of paragraphs A2 through A2.1.1, wherein determining the physical readiness indicator comprises assigning the heart rate variability data to a first physical readiness indicator of a set of two or more physical readiness indicators based on the comparison of the collected heart rate variability data with the one or more threshold physical readiness values.
A3. The method of any of paragraphs A0 through A2.1.1, wherein dynamically altering the workout in response to the determined physical readiness indicator of the user comprises altering one or more output parameters.
A3.1. The method of paragraph A3, wherein at least one of the one or more output parameters comprises a workout intensity.
A3.2. The method of paragraph A3 or A3.1, wherein at least one of the one or more output parameters comprises a workout duration.
A3.3. The method of any of paragraphs A3 through A3.2, wherein altering at least one of the one or more output parameters comprises decreasing the at least one of the one or more output parameters.
A4. The method of any of paragraphs A0 through A3.3, wherein the workout comprises an endurance sport, optionally cycling, running, or swimming.
A5. The method of any of paragraphs A0 through A4, wherein the heart rate variability data comprises root-mean-square of successive differences data.
A5.1. The method of any of paragraphs A0 through A4, wherein the heart rate variability data comprises standard deviation of normal-to-normal intervals data.
A6. The method of any of paragraphs A0 through A5.1, the method comprising periodically reassessing the physical readiness indicator and further adjusting the one or more workout outputs accordingly.
B0. A method of adjusting one or more workout outputs, the method comprising: receiving, by a user interface, one or more workout inputs provided by a user; generating one or more workout outputs based on the one or more workout inputs;
   based on a physical readiness category of the user, dynamically altering at least one workout output of the one or more workout outputs; and
   providing the one or more workout outputs to the user.
B1. The method of paragraph B0, further comprising comparing a physical readiness of the user to one or more threshold physical readiness levels; and assigning the physical readiness category of the user based on the comparison of the physical readiness of the user to the one or more threshold physical readiness levels.
B1.1. The method of paragraph B1, further comprising normalizing heart rate variability data to a numerical scale indicating the physical readiness of a user.
B1.2. The method of paragraph B1.1., wherein the normalizing the collected heart rate variability data to the numerical scale is performed by a/the heart rate measurement device.
B1.3. The method of any of paragraphs B1 through B1.2, wherein the threshold heart rate variability levels correspond to points on the numerical scale.
B2. The method of any of paragraphs B0, further comprising collecting the heart rate variability data of a user.
B2.1. The method of paragraph B2, wherein collecting the heart rate variability data of the user comprises obtaining the heart rate variability data from a heart rate measurement device.
B2.2. The method of paragraph B2.1, wherein the heart rate measurement device comprises a heart rate sensor operatively coupled to a processor configured to calculate heart rate variability data from data collected by the heart rate sensor.
B3. The method of any of paragraphs B0 through B2.2, wherein the physical readiness category is a member of a set of two or more physical readiness categories.
B4. The method of any of paragraphs B0 through B3, wherein dynamically altering the workout based on the assigned physical readiness category comprises altering one or more workout parameters.
B4.1. The method of paragraph B4, wherein at least one of the one or more workout parameters comprises a workout intensity.
B4.2. The method of paragraph B4 or B4.1, wherein at least one of the one or more workout parameters comprises a workout duration.
B4.3. The method of any of paragraphs B4 through B4.2, wherein altering at least one of the one or more workout parameters comprises decreasing the at least one of the one or more workout parameters.
B5. The method of any of paragraphs B0 through B4.3, wherein the workout comprises an endurance sport, optionally cycling, running, or swimming.
B6. The method of any of paragraphs B0 through B5, wherein the heart rate variability data comprises root-mean-square of successive differences data.
B6.1. The method of any of paragraphs B0 through B5, wherein the heart rate variability data comprises standard deviation of normal-to-normal intervals data.
B7. The method of any of paragraphs B0 through B6.1, the method comprising periodically reassessing the physical readiness indicator and further adjusting the one or more workout outputs accordingly.
C0. A system for automatically adjusting a workout based on heart rate variability, the system comprising:
   a wearable heart rate monitor; and
   an adjustment module configured to perform any of the methods of paragraphs A0 through B6.1 based on heart rate variability data collected by the wearable heart rate monitor.
D0. Non-transitory computer-readable storage media comprising computer executable software that when executed, direct a computing device to perform the method of any of paragraphs A0-B7.
E0. A product comprising any feature described herein, either individually or in combination with any other such feature, in any configuration.
F0. A process for automatically adjusting a workout based on heart rate variability data, the process comprising any process step described herein, in any order, using any modality.

### Advantages, Features, and Benefits

The different embodiments and examples of the system for automatically adjusting a workout based on heart rate variability data described herein provide several advantages over known solutions for generating workout regimens. For example, illustrative embodiments and examples described herein provide a groundbreaking approach to optimize athletic training by integrating HRV insights with artificial intelligence methods. Additionally or alternatively, the system for automatically adjusting a workout based on heart rate variability data described herein ensures that athletes receive personalized exercise prescriptions that dynamically adapt to their daily physical readiness. No known system or device can perform these functions. However, not all embodiments and examples of the system for automatically adjusting a workout based on heart rate variability data described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A method of adjusting one or more workout outputs, the method comprising:
receiving, by a user interface, one or more workout inputs provided by a user;
generating the one or more workout outputs based on the one or more workout inputs;
dynamically altering at least one of the one or more workout outputs based on a determined physical readiness indicator of the user; and
displaying the one or more workout outputs to the user.

2. The method of claim 1, further comprising determining a physical readiness indicator of the user based on heart rate variability data.

3. The method of claim 2, further comprising obtaining the heart rate variability data from a heart rate measurement device comprising a heart rate sensor operatively coupled to a processor configured to calculate the heart rate variability data from data collected by the heart rate sensor.

4. The method of claim 2, wherein the determining the physical readiness indicator of the user further comprises:
comparing the heart rate variability data with one or more threshold values; and
determining the physical readiness indicator based on a comparison of the heart rate variability data with the one or more threshold values.

5. The method of claim 4, wherein the determining the physical readiness indicator of the user comprises normalizing the heart rate variability data to a numerical scale.

6. The method of claim 4, wherein determining the physical readiness indicator comprises assigning the heart rate variability data to a first physical readiness indicator of a set of two or more physical readiness indicators based on the comparison of the heart rate variability data with the one or more threshold values.

7. The method of claim 1, wherein dynamically altering the one or more workout outputs in response to the determined physical readiness indicator of the user comprises adjusting one or more output parameters.

8. The method of claim 7, wherein at least one of the one or more output parameters comprises a workout intensity.

9. The method of claim 7, wherein the adjusting at least one of the one or more output parameters comprises decreasing the at least one of the one or more output parameters.

10. The method of claim 1, the method comprising periodically reassessing the determined physical readiness indicator and further adjusting the one or more workout outputs accordingly.

11. Non-transitory computer-readable storage media comprising computer executable software that when executed, direct a computing device to perform steps comprising:
receiving, by a user interface, one or more workout inputs provided by a user;
generating one or more workout outputs based on the one or more workout inputs;
dynamically altering at least one workout output of the one or more workout outputs based on a physical readiness category of the user; and
providing the one or more workout outputs to the user.

12. The non-transitory computer-readable storage media comprising computer executable software of claim 11, wherein the steps further comprise comparing a physical readiness of the user to one or more threshold physical readiness levels; and
assigning the physical readiness category of the user based on a comparison of the physical readiness of the user to the one or more threshold physical readiness levels.

13. The non-transitory computer-readable storage media comprising computer executable software of claim 12, wherein the steps further comprise normalizing heart rate variability data to a numerical scale indicating the physical readiness of a user.

14. The non-transitory computer-readable storage media comprising computer executable software of claim 13, wherein the threshold physical readiness levels correspond to points on the numerical scale.

15. The non-transitory computer-readable storage media comprising computer executable software of claim 11, wherein the physical readiness category is a member of a set of two or more physical readiness categories.

16. The non-transitory computer-readable storage media comprising computer executable software of claim 11, wherein the dynamically altering the at least one workout output of the one or more workout outputs based on the physical readiness category of the user comprises adjusting one or more workout parameters.

17. The non-transitory computer-readable storage media comprising computer executable software of claim 16, wherein the one or more workout parameters comprises a workout intensity.

18. The non-transitory computer-readable storage media comprising computer executable software of claim 16, wherein the adjusting the one or more workout parameters comprises decreasing at least one workout parameter of the one or more workout parameters.

19. The non-transitory computer-readable storage media comprising computer executable software of claim 11, wherein the steps further comprise periodically reassessing the physical readiness category and further altering the one or more workout outputs accordingly.

20. The non-transitory computer-readable storage media comprising computer executable software of claim 11, wherein the steps further comprise obtaining heart rate variability data from a heart rate measurement device, comprising a heart rate sensor operatively coupled to a processor configured to calculate the heart rate variability data from data collected by the heart rate sensor.
